(19) United States
(12) Patent Application Publication
(10) Pub. No.: US 2022/0145353 A1
(43) Pub. Date: May 12, 2022

(54) Sensitive glucose assay

(71) Applicant: Hoffmann-La Roche Inc., Little Falls, NJ (US)

(72) Inventors: Stephen FOWLER, Basel (CH); Na Hong QIU, Basel (CH)

(73) Assignee: Hoffmann-La Roche Inc., Little Falls, NJ (US)

(21) Appl. No.: 17/348,748

(22) Filed: Jun. 15, 2021

Related U.S. Application Data
(63) Continuation of application No. PCT/EP2019/085215, filed on Dec. 16, 2019.

(30) Foreign Application Priority Data
Dec. 17, 2018 (EP) 18213027.8

(51) Int. Cl. C12Q 1/54; G01N 33/66

(52) U.S. Cl. CPC C12Q 1/54; G01N 33/66

(57) ABSTRACT

The present invention provides a sensitive assay for determining the concentration of glucose in a sample and its applications in detecting enzymes converting a substrate to glucose.

### CROSS REFERENCE TO RELATED APPLICATIONS

This application is a continuation of International Application No. PCT/EP2019/085215, filed Dec. 16, 2019, which claims priority to European Patent Application No. 18213027.8, filed Dec. 17, 2018, which are incorporated herein by reference in its entirety.

The present invention provides a sensitive assay for determining the concentration of glucose in a sample and its applications in detecting enzymes converting a substrate to glucose.

Many glucose quantification methods are currently used to determine the glucose content. Among these, the most sensitive Amplex red glucose assay can detect glucose at the level >=3 μM. It is still not sensitive enough for samples, which are derived from some reactions such as Glucocerobrosidase assay, with the glucose concentration below 1 μM. Therefore, there is a need for a sensitive assay for determining glucose concentration in a sample.

The present invention provides a method for determining the concentration of glucose in a sample comprising the steps:
a) providing a liquid sample with glucose in a reaction tube,
b) oxidation of the glucose in the liquid sample of step a) and thereby producing H₂O₂,
c) providing a reaction tube coated with a protein, comprising a solution comprising a peroxidase enzyme and tyramide conjugated to a first member of a binding pair, and transferring the resulting solution of step b) to the reaction tube of step c) and thereby activating the conjugated tyramide which binds to the coated protein,
d) adding an enzyme conjugated to a second member of the binding pair to the solution of step c) and allow binding of the conjugated enzyme to the conjugated tyramide through interaction of the first and second member of the binding pair,
e) add a substrate for the conjugated enzyme to the solution of step e), wherein the conjugated enzyme converts the substrate to a compound with a measurable readout,
f) measuring the readout in the mixture of step e) and
g) converting the measured readout to glucose concentration.

In an embodiment of the invention, the first member of the binding pair is biotin and the second member of the binding pair is streptavidin.

In an embodiment of the invention, the glucose oxidation in step b) is an enzymatic oxidation by glucose oxidase.

In an embodiment of the invention, the peroxidase enzyme in step b) is horseradish peroxidase.

In an embodiment of the invention, the conjugated enzyme in step d) is alkaline phosphatase.

In an embodiment of the invention, the measurable readout in step d) is a colorimetric readout.

In an embodiment of the invention, the glucose sample is a body fluid sample, preferably a plasma or serum sample.

In an embodiment of the invention, the peroxidase enzyme in step c) is bound to the wall of the reaction tube.

In an embodiment of the invention, the method is performed in a multi well plate, preferably a 96 well plate, more preferably a MaxiSorp™ plate.

In an embodiment of the invention, the reaction tube in step c) is coated with BSA.

In an embodiment of the invention, the multi well plate is washed after step c) to remove unbound conjugated tyramide.

In an embodiment of the invention, the multi well plate is washed after step d) to remove unbound conjugated enzyme.

In an embodiment of the invention, the resulting solution of step e) is transferred to a multi well plate to measure the signal readout, preferably an IMAPlate™.

In an embodiment of the invention, the method is performed at 20° C. (room temperature).

In a second aspect the present invention provides a method for the determination of Glucocerobrosidase enzyme concentration in a sample comprising the steps:
a) providing a sample with Glucocerebrosidase.
b) adding a substrate of Glucocerebrosidase to the sample of step a) thereby generating glucose,
c) determining the glucose concentration in the resulting mixture of step b) using a method of the present invention and
d) converting the glucose concentration to Glucocerobrosidase concentration.

In an embodiment of the invention, the Glucocerobrosidase substrate is glucosylceramide.

In an embodiment of the invention, the sample is a body fluid sample, preferably a plasma or serum sample.

The present invention provides a sensitive assay for determining the concentration of glucose as low as 0.005 μM. In the invention, glucose, glucose oxidase and horseradish peroxidase activate the biotinylated tyramide, resulting biotinylated tyramide deposits to immobilized protein; when addition of streptavidin conjugated alkaline phosphatase, alkaline phosphatase can tightly bind to biotinylated tyramide and catalyze its substrate such as pNPP to form a product which is capable to be quantified by a spectrophotometer. Therefore, from glucose to the final pNPP product is not a 1:1 stoichiometry reaction; an enzyme amplification process is involved.

### SHORT DESCRIPTION OF THE FIGURES

FIG. 1 is a schematic overview of the chemical reactions of the method of the present invention.

FIG. 2 shows a glucose standard curve generated by using the method of the present invention. Buffer=PBS.

FIG. 3 shows a glucose standard curve generated by using the method of the present invention. Buffer=MES.

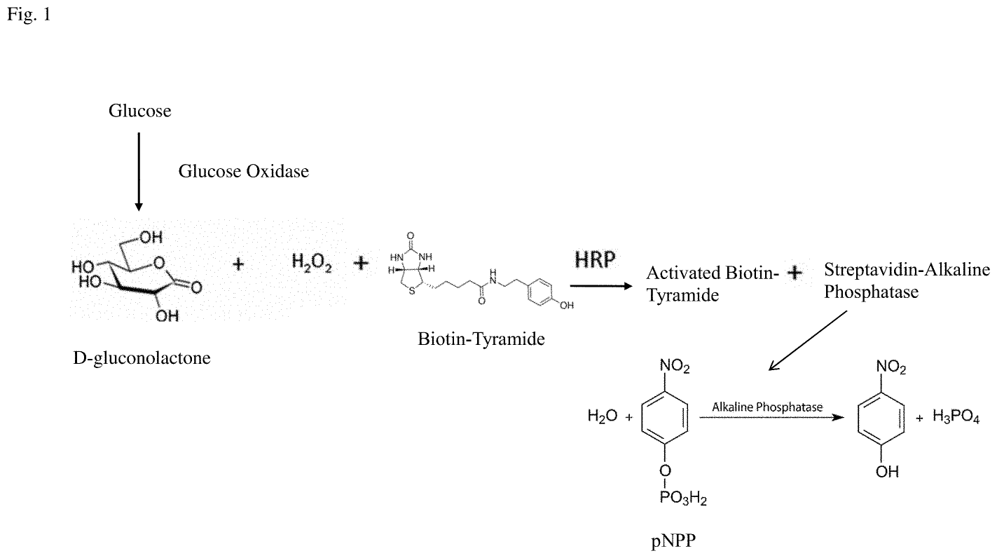

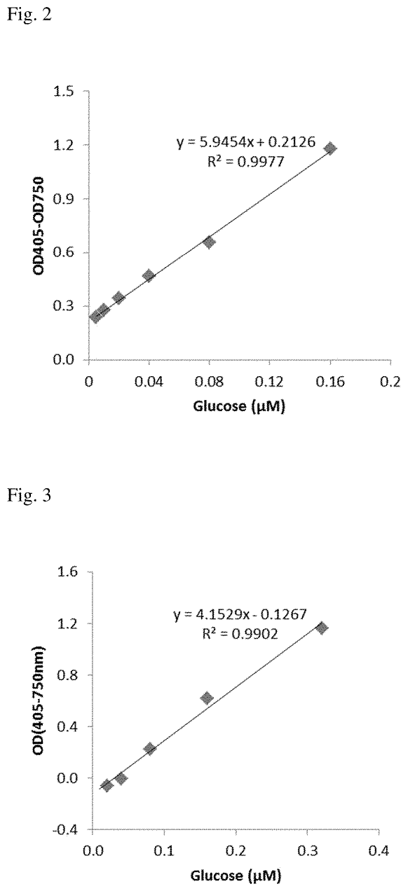

The term “peroxidase” is used herein to denote an enzyme that typically catalyzes a reaction of the form: ROOR′+electron donor (2 e−)+2H+-ROH+R′OH. A peroxidase that can be used in the methods described herein is capable of using a biotin tyramide compound, also known as biotin phenol, as a substrate, and converting it to a highly reactive free radical that binds covalently to electron-rich amino acids, resulting in their biotinylation. The chemical principles of tyramide reaction and its applications in protein labelling methods are described in U.S. Pat. No. 5,731,158 and McKay et al., “Amplification of fluorescent in situ hybridization signals in formalin fixed paraffin wax embedded sections of colon tumor using biotinylated tyramide,” J. Clin. Pathol: Mol. Pathol. 50:322-25, 1997. A peroxidase that can be used in the methods described herein can be a naturally occurring, modified, synthetic or engineered peroxidase.

The term “glucose oxidase (GOD)” is used herein to denote an enzyme which catalyzes the oxidation of β-d-glucose to d-glucono-δ-lactone and H₂O₂ using molecular oxygen as an electron acceptor. d-glucono-δ-lactone is then non-enzymatically hydrolyzed to gluconic acid. A glucose oxidase that can be used in the methods described herein can be a naturally occurring, modified, synthetic or engineered glucose oxidase.

### EXAMPLES

### Example 1

1. Plate coating: Add 100 μL the mixture of 1 μg/mL HRP and 1 μg/mL BSA (in PBS) into each well of a 96 well plate at RT, 2 hrs.
2. Wash the plate 3 times with 150 μL/well of washing buffer (PBS+0.05% Tween 20).
3. Prepare TSA reagent: 4 μg/mL glucose oxidase and 2 μM Biotin-tyramide in PBS.
4. Load to each well: 50 μL/well of TSA reagent plus 50 μL/well of glucose (in PBS or other matrix) standards (typical concentration: 0.32, 0.16, 0.08, 0.04, 0.02, 0.01 and 0.005 μM), blank (50 μL of PBS) and 50 μL/well of test samples. Mix and incubation at RT, 20 mins.
5. Wash the plate 6 times with 150 μL/well of washing buffer (PBS+0.05% Tween 20) to remove inactivated (non-deposition) biotin-tyramide.
6. Add 100 μL/well of streptavidin-alkaline phosphatase to each well and incubate at RT for 15 mins.
7. Wash the plate 6 times with 150 μL/well of washing buffer (PBS+0.05% Tween 20) to remove unbounded alkaline phosphatase.
8. Add 50 μL/well of alkaline phosphatase substrate pNPP and incubate ˜20 mins at RT with shaking set the speed at 450 rpm, transfer 30 μL to 96 well IMAPlate for results readout (using plate reader set wavelength at 405 nm and reference wavelength at 750 nm).

Material

96 well plate (Nunc Clear U-Bottom Immuno plate, MaxiSorp™)

Horseradish peroxidase (HRP)

Bovine serum albumin (BSA)

Phosphate-buffered saline (PBS)

Glucose oxidase (GOD)

Biotin-tyramide

D-Glucose standard

Streptavidin-alkaline phosphatase (streptavidin-AP)

pNPP (para-Nitrophenylphosphat)

96 well IMAPlate™ white

Tween-20

1. A method for determining the concentration of glucose in a sample comprising the steps:
a) providing a liquid sample with glucose in a reaction tube,
b) oxidation of the glucose in the liquid sample of step a) and thereby producing H₂O₂,
c) providing a reaction tube coated with a protein, comprising a solution comprising a peroxidase enzyme and tyramide conjugated to a first member of a binding pair, and transferring the resulting solution of step b) to the reaction tube of step c) and thereby activating the conjugated tyramide which binds to the coated protein,
d) adding an enzyme conjugated to a second member of the binding pair to the solution of step c) and allow binding of the conjugated enzyme to the conjugated tyramide through interaction of the first and second member of the binding pair,
e) add a substrate for the conjugated enzyme to the solution of step e), wherein the conjugated enzyme converts the substrate to a compound with a measurable readout,
f) measuring the readout in the mixture of step e) and
g) converting the measured readout to glucose concentration.

2. The method of claim 1, wherein the first member of the binding pair is biotin and the second member of the binding pair is streptavidin.

3. The method of claim 1, wherein the glucose oxidation in step b) is an enzymatic oxidation by glucose oxidase.

4. The method of claim 1, wherein the peroxidase enzyme in step b) is horseradish peroxidase.

5. The method of claim 1, wherein the conjugated enzyme in step d) is alkaline phosphatase.

6. The method of claim 1, wherein the measurable readout in step d) is a colorimetric readout.

7. The method of claim 1, wherein the glucose sample is a body fluid sample, wherein the body fluid sample is a plasma or serum sample.

8. The method of claim 1, wherein the peroxidase enzyme in step c) is bound to the wall of the reaction tube.

9. The method of claim 1, wherein the method is performed in a multi well plate.

10. The method of claim 1, wherein the reaction tube in step c) is coated with BSA.

11. The method of claim 9, wherein the multi well plate is washed after step c) to remove unbound conjugated tyramide.

12. The method of claim 9, wherein the multi well plate is washed after step d) to remove unbound conjugated enzyme.

13. The method of claim 9, wherein the resulting solution of step e) is transferred to a multi well plate to measure the signal readout.

14. The method of claim 1, wherein the method is performed at 20° C. or at room temperature.

15. A method for the determination of Glucocerobrosidase enzyme concentration in a sample comprising the steps:
a) providing a sample with Glucocerebrosidase.
b) adding a substrate of Glucocerebrosidase to the sample of step a) thereby generating glucose,
c) determining the glucose concentration in the resulting mixture of step b) using the method of claim 1, and
d) converting the glucose concentration to Glucocerobrosidase concentration.

16. The method of claim 15, wherein the Glucocerobrosidase substrate is glucosylceramide.

17. The method of claim 15, wherein the sample is a body fluid sample, wherein the body fluid is a plasma or serum sample.
